(19) United States
(12) Patent Application Publication
(10) Pub. No.: US 2016/0038771 A1
(43) Pub. Date: Feb. 11, 2016

(54) HANDPIECE FOR ULTRASONIC DEVICE

(71) Applicant: CLASSYS INC., Seoul (KR)

(72) Inventor: Sung Jae JUNG, Seoul (KR)

(21) Appl. No.: 14/779,950

(22) PCT Filed: Sep. 9, 2013
(86) PCT No.: PCT/KR2013/008101

§ 371 Date: Sep. 24, 2015

(30) Foreign Application Priority Data
Mar. 26, 2013 (KR) 10-2013-0032374

(51) Int. Cl. A61N 7/02

(52) U.S. Cl. CPC A61N 7/02; A61N 2007/0034

(57) ABSTRACT

A handpiece for an ultrasonic device includes a handpiece main body and a detachable cartridge attached to the handpiece main body. The handpiece main body includes a main body housing, an actuator for axially transferring a transducer, an operating button, and a handpiece main substrate for operating the actuator according to the control of a controller unit, supplying power to the transducer according to on/off operations of the operating button, and receiving a signal detected by the transducer and transmitting the signal to the controller unit. The cartridge includes a cartridge housing, a transducer for generating ultrasound, a guide pin for guiding the transducer, a magnet, a slider for axially moving the transducer along the guide pin, a window for transferring the ultrasound to a patient, and a bellows for isolating liquid contained in the cartridge housing and helping easy transfer of the slider.

### TECHNICAL FIELD

The present invention generally relates to a treatment and diagnosis device using ultrasonic waves and, more particularly, to a monoaxial handpiece of a treatment and diagnosis device using high intensity focused ultrasound (HIFU).

### BACKGROUND ART

Recently, a meat-based diet has caused a sudden increase in the obesity of people, which has become a major factor affecting peoples' health and beauty. A variety of diet programs and ultrasonic obesity treatments have thus been widely developed and used in order to solve this problem.

As an obesity treatment, a high intensity focused ultrasound (FIFU) technique, which was originally used in cancer treatment by non-invasively performing selective high-temperature coagulation of internal organs tumors to destroy cancer cells, is often used. In fact, an HIFU-equipped device named ‘Liposonix’ was been first developed by Solta Medical, Co., Ltd. in the USA as an abdominal obesity-treatment device. Fat-destruction using HIFU is performed by focusing ultrasound on a specified site of fat cells so that the temperature of the tissue suddenly rises up to 65° C. to 100° C., thereby destroying the tissue. Unlike other dermatology equipment, such as a laser, or RF equipment, an HIFU device can cause coagulation necrosis of fat tissue by non-invasive focusing of HIFU energy on a selective site without any damage on the skin surface. The dead fat cells are naturally removed by a body's restoration mechanism.

As a known obesity treatment technique using ultrasound, a device and method for destroying fat tissue are disclosed in Korean Patent Application No. 10-2007-7027898. The device disclosed in Korean Patent Application No. 10-2007-7027898 includes a scan head having at least an ultrasonic converter, a suspension unit for supporting the scan head, and a controller for controlling the ultrasonic converter and is configured to non-invasively treat obesity by deforming subcutaneous fat tissue with ultrasonic energy. Further, as another technique, a tissue imaging and treating system disclosed in Korean Patent Application No. 10-2011-7017983 includes a hand wand activated by hand, a detachable emitter-receiver module having an ultrasonic transducer, and an interface connected with the hand wand such that in imaging mode, a skin image is displayed on a display, and in treatment mode, an ultrasonic wave is emitted towards subcutaneous fat tissue (or SMAS layer) 3 mm to 4.5 mm under the skin surface to improve skin elasticity and prevent skin wrinkles.

Such ultrasonic obesity treatment devices include a head or a handpiece to generate HIFU under the control of a controller and scan subcutaneous fat tissue of a patient. A therapy head for use in HIFU procedures is disclosed in Korean Patent Application No. 10-2006-7013272. The therapy head has an enclosure with a window, an HIFU transducer suspended from the inside of the enclosure, and an actuator for moving the HIFU transducer along an x-axis and a y-axis to allow the HIFU transducer to scan subcutaneous fat tissue with HIFU through the window. Further, A therapy head using an ultrasonic system disclosed in Korean Patent Application No. 10-2010-7019552 includes an enclosure, a partition separating a lower compartment from an upper compartment, an aperture in the partition, a control arm extending through the aperture, an actuation assembly positioned within the upper compartment so as to control the control arm, and an HIFU transducer coupled with the lower side of the control arm.

### DISCLOSURE

### Technical Problem

A conventional handpiece for an ultrasonic device has a problem in that ultrasound is focused in a point type array by the transducer, which requires multi-axis (e.g. two or more axes) control, causing a complicated control algorithm and a prolonged operation time.

Accordingly, the present invention has been made keeping in mind the above problems occurring in the related art, and an object of the present invention is to provide an ultrasonic handpiece capable of treating skin and beauty at high speed in a monoaxial control manner by a line-focusing structure transducer.

### Technical Solution

In order to accomplish the above object, the present invention provides a handpiece for an ultrasonic device, including a handpiece main body and a cartridge attachable to and detachable from the handpiece main body. The handpiece main body includes: a main body housing; an actuator, installed in the main body housing, for axially transferring a transducer; an operating button mounted on the main body housing; and a handpiece main substrate for operating the actuator according to the control of a controller unit, supplying and cutting-off power to the transducer according to on/off operations of the operating button, and receiving, as an input, a signal detected by the transducer and transmitting the signal to the controller unit.

The cartridge includes: a cartridge housing; a transducer built in the cartridge housing for generating point-focused or line-focused ultrasound; a guide pin for axially guiding the transducer; a magnet for power transmission for transferring the power of the actuator to the cartridge; a slider for axially moving the transducer along the guide pin by the operation of the actuator; a window formed on one side of the cartridge housing for transferring the ultrasound generated by the transducer to a patient; and a bellows for isolating liquid contained in the cartridge housing and helping easy transfer of the slider.

The cartridge and the handpiece main body transfer the power of the actuator by the operation of the magnet for power transmission and connect a power supply and signals via a connector.

The handpiece main body further includes an LED substrate mounted in the main body housing for displaying the operating states, and the handpiece main substrate displays the operating states by turning on LEDs of the LED substrate.

The transducer includes a transducer housing, and a cylindrical piezoelectric element that is provided on one side of the transducer housing to produce ultrasound focused in a line type array, so that, when transferred along one axis, linear heat lines are formed on a 2D plane, or a spherical piezoelectric element that is provided on one side of the transducer housing to produce ultrasound focused in a point type array, so that, when transferred along one axis, heat points are formed on a 2D plane.

### Advantageous Effects

According to the present invention, the handpiece for an ultrasonic device can focus ultrasound in a line type array so that a 2D planar ultrasonic focusing area is formed only with the monoaxial control, providing easy manipulation and shortening the operating time.

Further, according to the present invention, the handpiece for an ultrasonic device can distribute a strong energy from the line-focusing transducer in a line type array while sequentially moving the transducer with the operation of the actuator under the control of the controller unit, thereby irradiating energy in a sheet form onto the skin, maximizing lifting effect of the skin and additionally facilitating an operation effective to removal of freckles, pigmentation, pimples, and to treat excessive sweating, osmidrosis, etc.

### DESCRIPTION OF DRAWINGS

FIG. 1 is a schematic perspective view of an ultrasonic treatment device to which a handpiece is adapted according to an embodiment of the present invention.

FIG. 2 is a perspective view of the handpiece according to the present invention.

FIG. 3 is a perspective view of the handpiece with a cartridge removed therefrom.

FIG. 4 is a side cross-sectional view of the handpiece.

FIG. 5 illustrates an exemplary line-focusing type transducer adapted to the handpiece according to an embodiment of the present invention.

FIG. 6 illustrates an exemplary point-focusing type transducer adapted to the handpiece according to an embodiment of the present invention.

FIG. 7 is a view showing the first operation of the handpiece according to the present invention.

FIG. 8 is a view showing the second operation of the handpiece according to the present invention.

FIG. 9 is a view showing the third operation of the handpiece according to the present invention.

### MODE FOR INVENTION

The technical problem of the present invention will become more apparent with reference to exemplary embodiments in the following description. The embodiments described are merely illustrative for describing the present invention, but are not intended to limit the scope of the present invention.

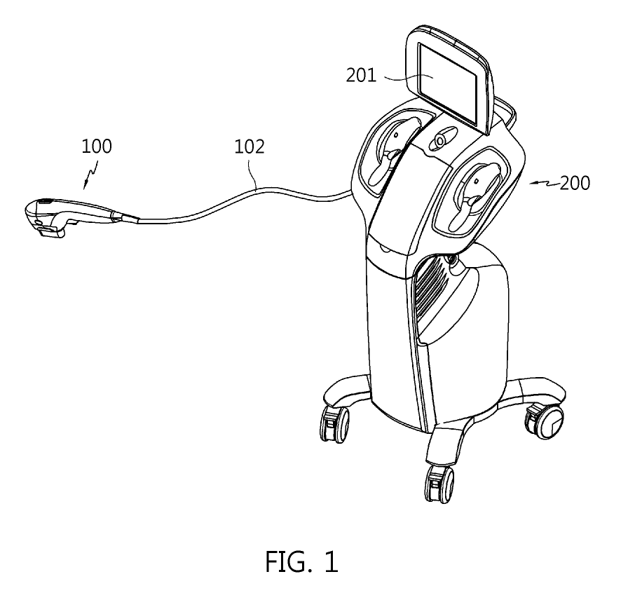

FIG. 1 is a schematic perspective view of an ultrasonic treatment device to which a handpiece according to the present invention is adapted.

As shown in FIG. 1, the handpiece 100 is connected with a controller unit 200 for an ultrasonic device having a touch screen 210 so as to treat and diagnose the skin of a patient according to the control of the controller unit 200.

Referring to FIG. 1, the controller unit 200 for an ultrasonic device has rollers for easy movement and is provided with an operating program for management of patients and an application program for diagnosis and treatment of the skin of a patient. In use, an internal controller executes the program to display an image obtained from the handpiece 100 on the touch screen 210 and to control entire operations with various inputs.

The handpiece 100 is connected with the controller unit 200 via cables 102, so that the handpiece can distribute a strong energy from a line-focusing type transducer in a line type array while sequentially moving the transducer with the operation of an actuator under the control of the controller unit 200, thereby irradiating energy in a sheet form onto the skin, maximizing lifting effect of the skin and additionally facilitating an operation effective to removal of freckles, pigmentation, pimples, and to treat excessive sweating, osmidrosis, etc.

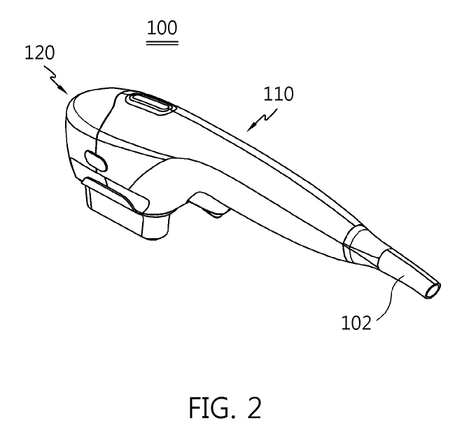

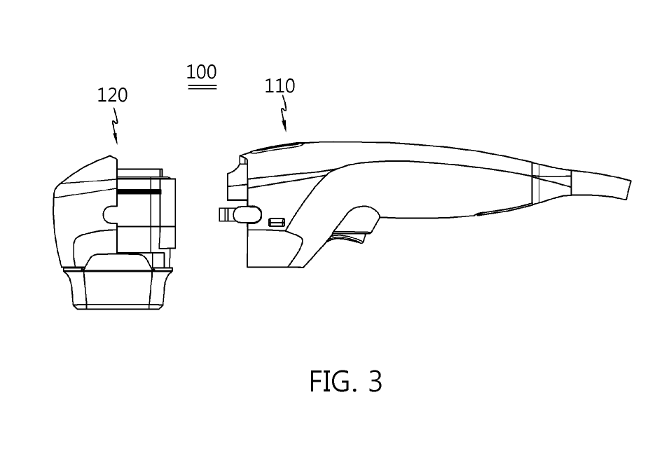

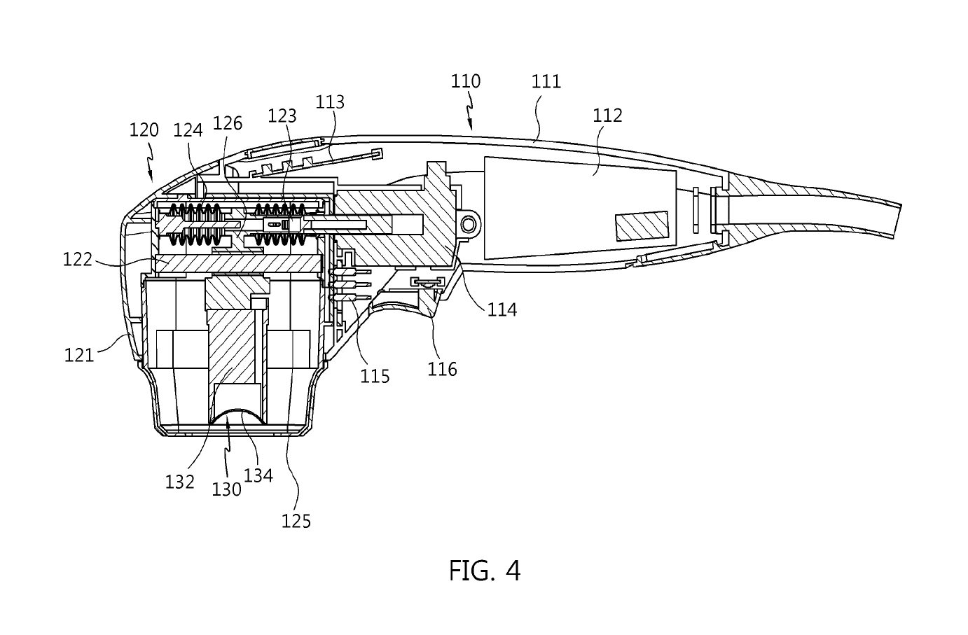

FIG. 2 is a perspective view of the handpiece according to the present invention. FIG. 3 is a perspective view of the handpiece with a cartridge removed therefrom, and FIG. 4 is a side cross-sectional view of the handpiece.

As shown in FIGS. 2 to 4, the handpiece 100 includes a cartridge 120 to be brought into contact with a portion of a user's face desired to be lifted, and a handpiece main body 110 attaching and driving the cartridge. The handpiece main body 110 is connected with the control unit 200 of the ultrasonic device via the cable 102. An actuator 114-axis and a cartridge-driving axis of the handpiece main body 110 are connected by a magnet 123 for power transmission, and electrical signals are connected via a connector 115.

Referring to FIGS. 2 to 4, the handpiece 100 includes the handpiece main body 100 and the cartridge 120 attachable to and detachable from the handpiece main body 110. The handpiece main body 110 includes a main body housing 111, a linear actuator 114 installed in the main body housing 111 for axially transferring a transducer 130; an LED substrate 113 installed on the main body housing 111 for displaying the operating states, an operating button 116, and a handpiece main substrate 112 for operating the actuator 114 according to the control of the controller unit 200, supplying and cutting-off power to the transducer 130 according to on/off operations of the operating button 116, receiving, as an input, a signal detected by the transducer 130 and transmitting the signal to the controller unit 200, and turning on LEDs of the LED substrate 113 to display the operating states.

The cartridge 120 includes a cartridge housing 121, a transducer 130 built in the cartridge housing 121 for generating point-focused or line-focused ultrasound, a guide pin 122 for axially guiding the transducer 130, a magnet 123 for power transmission for transferring the power of the actuator to the cartridge, a slider 126 for axially moving the transducer 130 along the guide pin 122 by the operation of the actuator, a window 125 formed on one side of the cartridge housing for transferring the ultrasound generated by the transducer 130 to a patient, and a water-tight bellows 124 for isolating liquid contained in the cartridge housing and helping easy transfer of the slider 126.

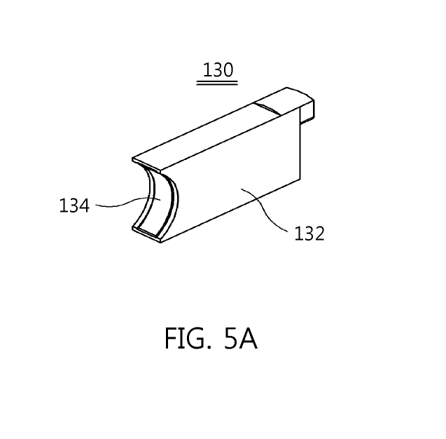

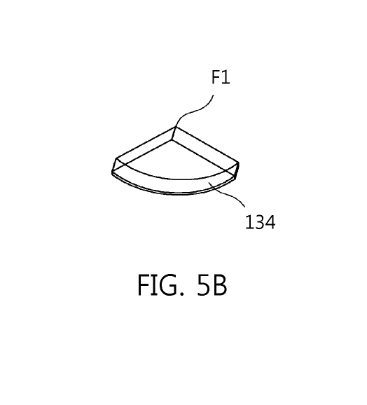

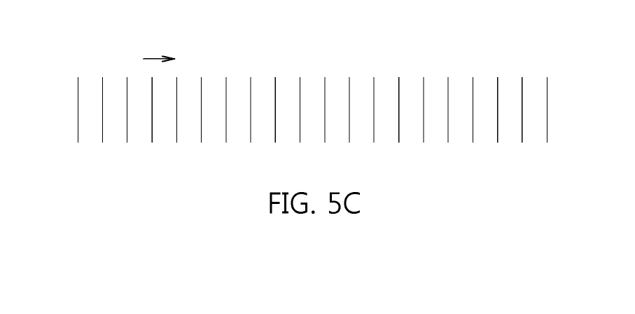

FIG. 5 illustrates an exemplary line-focusing type transducer 130 adapted to the handpiece according to an embodiment of the present invention, wherein FIG. 5 (a) shows a perspective view of the transducer, FIG. 5 (b) shows an ultrasonic beam generated by a cylindrical piezoelectric element, and FIG. 5 (c) shows an exemplary heat line region formed on a 2D plane when moved along an axis.

As shown in FIG. 5 (a), the line-focusing type transducer 130 includes a transducer housing 132, and a cylindrical piezoelectric element 134 that is provided on one side of the transducer housing 132 to produce ultrasound focused in a line type array. As shown in FIG. 5 (b), the ultrasonic beam F1 formed by the cylindrical piezoelectric element 134 shows a linear form, so that, when transferred along one axis, a linear heat line is formed on a 2D plane as shown in FIG. 5 (c).

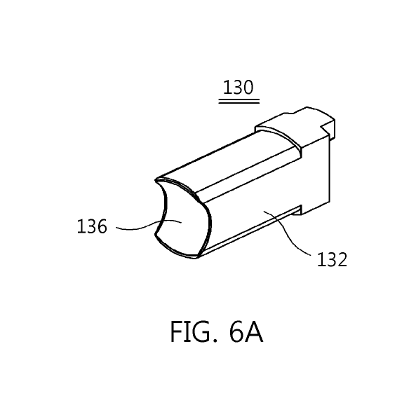

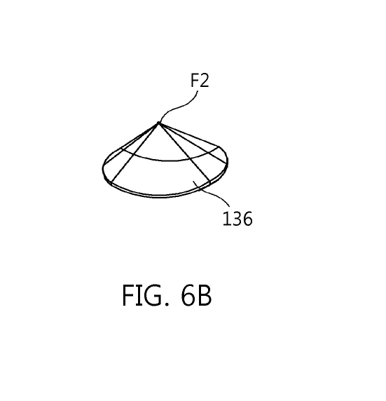

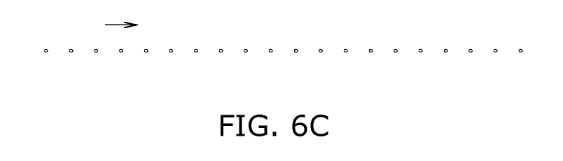

FIG. 6 illustrates an exemplary point-focusing type transducer 130 adapted to the handpiece according to an embodiment of the present invention, wherein FIG. 6 (a) shows a perspective view of the transducer, FIG. 6 (b) shows an ultrasonic beam generated by a spherical piezoelectric element, and FIG. 6 (c) shows an exemplary heat point region formed on a 2D plane when moved along an axis.

As shown in FIG. 6, the point-focusing type transducer 130 includes a transducer housing 132, and a spherical piezoelectric element 136 that is provided on one side of the transducer housing 132 to produce ultrasound focused in a point type array. As shown in FIG. 6 (b), the ultrasonic beam F2 formed by the spherical piezoelectric element 136 shows a point-like form, so that, when the handpiece 100 is transferred along one axis, heat points are formed on a 2D plane as shown in FIG. 6 (c).

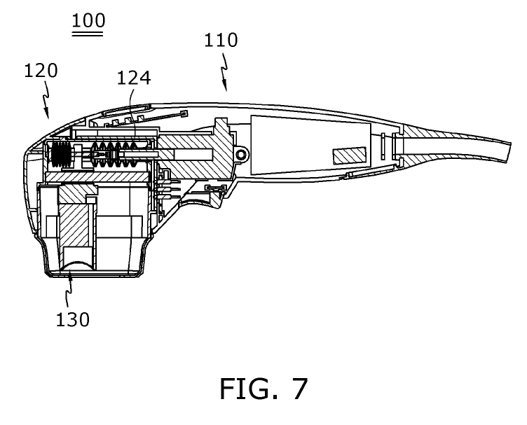

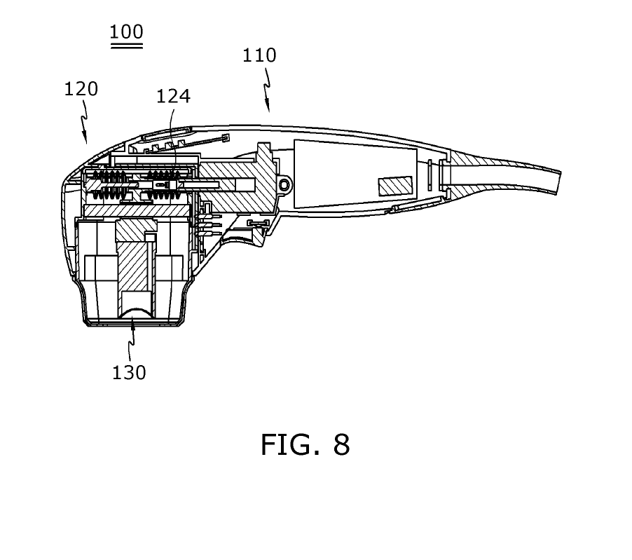

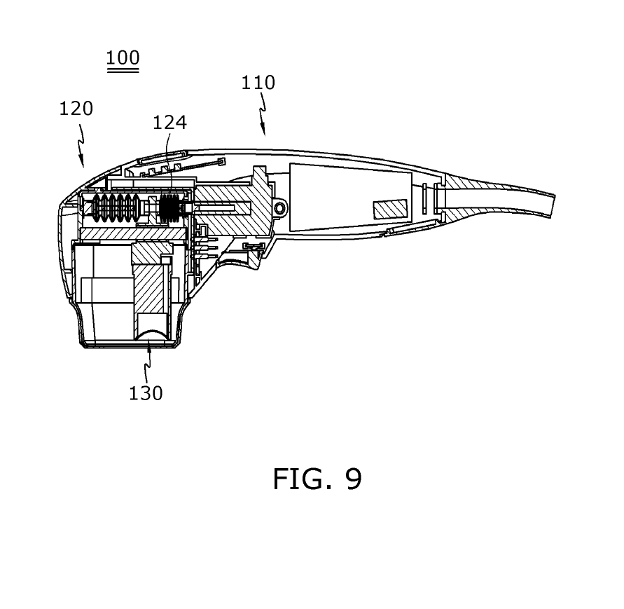

FIG. 7 is a view showing the first operation of the handpiece according to the present invention, FIG. 8 is a view showing the second operation of the handpiece according to the present invention, and FIG. 9 is a view showing the third operation of the handpiece according to the present invention.

In operation of the handpiece 100, when linearly moved according to the control of the controller unit 200, the linear actuator 114 is coupled to the magnet 123 for power transmission at the same axis as the slider 126 of the cartridge so that the transducer 130 moves horizontally to the left or right side according to the motion of the linear axis. When the transducer 130 moves to the left side, as shown in FIG. 7, the bellows 124 is also biased to the left side so that a left-side part thereof is compressed while a right-side part thereof is stretched. When the transducer is positioned at a middle position, as shown in FIG. 8, left and right side parts of the bellows 124 maintains an original state. When the transducer moves to the right side, as shown in FIG. 9, the right-side part of the bellows is compressed while the left-side part is stretched so that it is freely movable horizontally along the guide pin 122.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### DESCRIPTION OF REFERENCE SIGNS

100: Handpiece
200: Controller unit
110: Handpiece main body
120: Cartridge
210: Touch screen
102: Cable
111: Main body housing
112: Main substrate
113: LED substrate
114: Actuator
115: Power supply and signal line
116: Operation button
121: Cartridge housing
122: Guide pin
123: magnet for power transmission
124: Water-tight bellows
125: Window
126: Slider
130: Transducer
132: Transducer housing
134: Cylindrical piezoelectric element
136: Spherical piezoelectric element

1. A handpiece for an ultrasonic device, comprising:
a handpiece main body; and
a cartridge attachable to and detachable from the handpiece main body, the handpiece main body comprising:
a main body housing;
an actuator installed in the main body housing for axially transferring a transducer;
an operating button mounted on the main body housing; and
a handpiece main substrate for operating the actuator according to the control of a controller unit, supplying and cutting-off power to the transducer according to on and off operations of the operating button, and receiving, as an input, a signal detected by the transducer and transmitting the signal to the controller unit, the cartridge comprising:
a cartridge housing;
a transducer built in the cartridge housing for generating point-focused or line-focused ultrasound;
a guide pin for axially guiding the transducer;
a magnet for power transmission for transferring the power of the actuator to the cartridge;
a slider for axially moving the transducer along the guide pin by the operation of the actuator;
a window formed on one side of the cartridge housing for transferring the ultrasound generated by the transducer to a patient; and
a bellows for isolating liquid contained in the cartridge housing and helping easy transfer of the slider.

2. The handpiece for an ultrasonic device according to claim 1, wherein the cartridge and the handpiece main body transfer the power of the actuator by the operation of the magnet for power transmission and connect a power supply and signals via a connector.

3. The handpiece for an ultrasonic device according to claim 1, wherein the handpiece main body further includes an LED substrate mounted in the main body housing for displaying the operating states, and the handpiece main substrate displays the operating states by turning on LEDs of the LED substrate.

4. The handpiece for an ultrasonic device according to claim 1, wherein the transducer includes a transducer housing, and a cylindrical piezoelectric element that is provided on one side of the transducer housing to produce ultrasound focused in a line type array, so that, when transferred along one axis, linear heat lines are formed on a 2D plane, or a spherical piezoelectric element that is provided on one side of the transducer housing to produce ultrasound focused in a point type array, so that, when transferred along one axis, heat points are formed on a 2D plane.

5. The handpiece for an ultrasonic device according to claim 1, wherein the transducer includes a transducer housing, and a spherical piezoelectric element which is provided on one side of the transducer housing to produce ultrasound focused in a point type array, so that, when transferred along one axis, heat points are formed on a 2D plane.

6. The handpiece for an ultrasonic device according to claim 2, wherein the transducer includes a transducer housing, and a cylindrical piezoelectric element that is provided on one side of the transducer housing to produce ultrasound focused in a line type array, so that, when transferred along one axis, linear heat lines are formed on a 2D plane, or a spherical piezoelectric element that is provided on one side of the transducer housing to produce ultrasound focused in a point type array, so that, when transferred along one axis, heat points are formed on a 2D plane.

7. The handpiece for an ultrasonic device according to claim 3, wherein the transducer includes a transducer housing, and a cylindrical piezoelectric element that is provided on one side of the transducer housing to produce ultrasound focused in a line type array, so that, when transferred along one axis, linear heat lines are formed on a 2D plane, or a spherical piezoelectric element that is provided on one side of the transducer housing to produce ultrasound focused in a point type array, so that, when transferred along one axis, heat points are formed on a 2D plane.

8. The handpiece for an ultrasonic device according to claim 2, wherein the transducer includes a transducer housing, and a spherical piezoelectric element which is provided on one side of the transducer housing to produce ultrasound focused in a point type array, so that, when transferred along one axis, heat points are formed on a 2D plane.

9. The handpiece for an ultrasonic device according to claim 3, wherein the transducer includes a transducer housing, and a spherical piezoelectric element which is provided on one side of the transducer housing to produce ultrasound focused in a point type array, so that, when transferred along one axis, heat points are formed on a 2D plane.
